(19) United States
(12) Patent Application Publication
(10) Pub. No.: US 2006/0155171 A1
(43) Pub. Date: Jul. 13, 2006

(54) Illumination apparatus for oral cavity

(76) Inventor: Chien Hsin Yang, Sanchong City (TW)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(21) Appl. No.: 11/326,846

(22) Filed: Jan. 6, 2006

(30) Foreign Application Priority Data
Jan. 13, 2005 (TW) 94200637

(51) Int. Cl. A61B 1/32; A61C 1/00

(52) U.S. Cl. 600/238; 433/29

(57) ABSTRACT

An apparatus for illuminating an oral cavity having a generally U-shaped member having two ends and an intermediate portion defined therebetween. The apparatus can include generally arcuate braces defined at each of the ends and an illumination device operably coupled to at least one of the braces. The apparatus can further include an electric connecter operably coupled to the intermediate portion, the connecter being operably coupled to the illumination device with one or more wires. The illumination devices on the arcuate braces can sufficiently illuminate the patient's oral cavity during treatment.

### FIELD OF THE INVENTION

The present invention generally relates to an illumination apparatus for a patient's oral cavity. More particularly, the present invention relates to an illumination apparatus that can force an oral cavity open and illuminate the interior of the oral cavity.

### BACKGROUND TO THE INVENTION

With the promotion of medical treatment qualities, modem people begin to pay attention to qualities of various medical treatments, and particularly, dental treatments. However, some patients cannot open their mouths sufficiently during the course of treatment. Therefore, the dentist will force the patient's mouth to open to an extent that the treatment can be applied by using an oral expander.

Referring to FIGS. 5 and 6, a conventional oral expander has a generally U-shaped sheet 50 having arcuate braces 51 formed at each of the two free ends thereof. The generally U-shaped sheet 50 is generally made of elastic, plastic materials.

When in use, the two arcuate braces 51 are each placed at the corners of the patient's mouth to come into contact with the corners of the mouth. The elastic generally U-shaped sheet 50 expands toward both sides, and the arcuate braces 51 make the mouth expand toward both sides. Thus the patient's oral cavity is forced to open and cannot close so that the dentist can begin the treatment.

During the course of current dental treatment, the dentist will ask the patient to lie on a dental chair, use an illumination apparatus positioned above to illuminate the patient's oral cavity, and treat the teeth by using proper medical instruments. When treating the patient, the dentist sometimes will adjust his position relative to the patient for facilitating the treatment of the patient due to the lying angle of the patient.

However, when adjusting the position, the dentist often blocks the light that illuminates the patient's oral cavity, and the treatment of the patient is influenced. Although the dentist uses an oral expander for facilitating the treatment of some patients, yet the oral expander does not have any illumination device provided thereon. Therefore, the oral expander substantially has no practicability with regard to the illumination on the patient's oral cavity.

### SUMMARY OF THE INVENTION

In consideration of the drawback that the conventional oral expander cannot provide proper illumination on the patient's oral cavity, the inventor thus re-designs an illumination apparatus for oral cavity, which is fixedly provided with illumination devices on one side of arcuate braces of an oral expander. With the provision of said illumination devices, the patient's oral cavity can be moderately illuminated so that the patient can be well treated under the condition of sufficient illumination, as is an object of the present invention.

In order to achieve the above-mentioned object, the present invention provides an illumination apparatus for oral cavity, having a generally U-shaped sheet. The generally U-shaped sheet has arcuate braces formed at each of the two free ends thereof. Illumination devices are fixedly provided on at least one side of each arcuate brace. An electric connecter, which is connected with the illumination devices via wires, is fixedly provided at the closed end of the generally U-shaped sheet.

When the two arcuate braces are each placed at the corners of the mouth, the arcuate braces come into contact with the corners of the patient's mouth. The generally U-shaped sheet, which expands outwardly, makes the mouth expand toward both sides, and further forces the oral cavity to open. The illumination devices on the two arcuate braces can emit light to illuminate the patient's palatal and mandible at the same time. Therefore, the patient can be well treated under the condition of sufficient illumination even if lights from the upper portion are blocked due to the dentist's position.

### BRIEF DESCRIPTION OF THE DRAWINGS

FIG. 1 is a first perspective view of an illumination device according to a first embodiment of the present invention;

FIG. 2 is a second perspective view of the illumination device of FIG. 1;

FIG. 3 depicts the illumination device inserted in a patient's oral cavity;

FIG. 4 is a perspective view of an illumination device according to a second embodiment;

FIG. 5 is a first perspective view of an oral expander; and

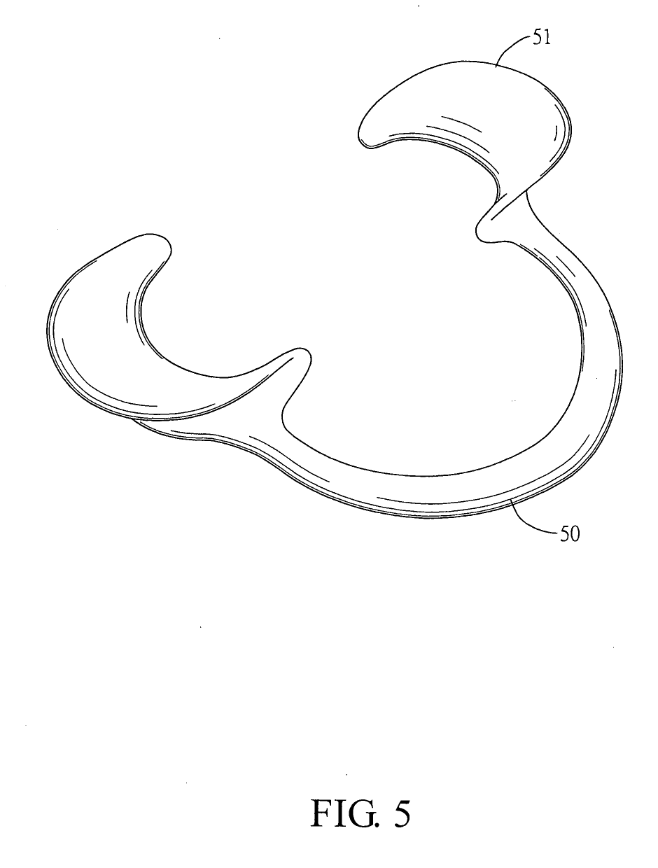

FIG. 6 is second perspective view of the oral expander of FIG. 5.

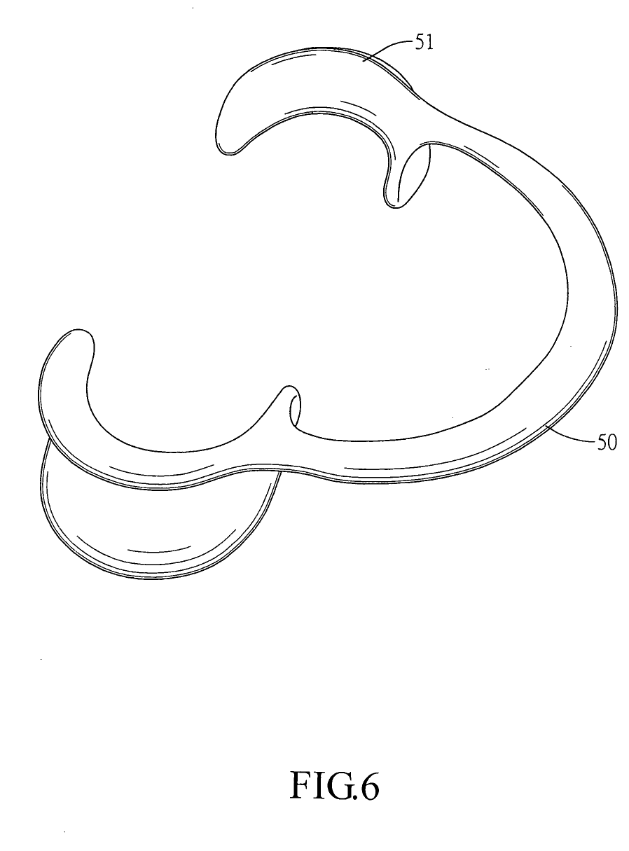

### DESCRIPTION OF PREFERRED EMBODIMENTS

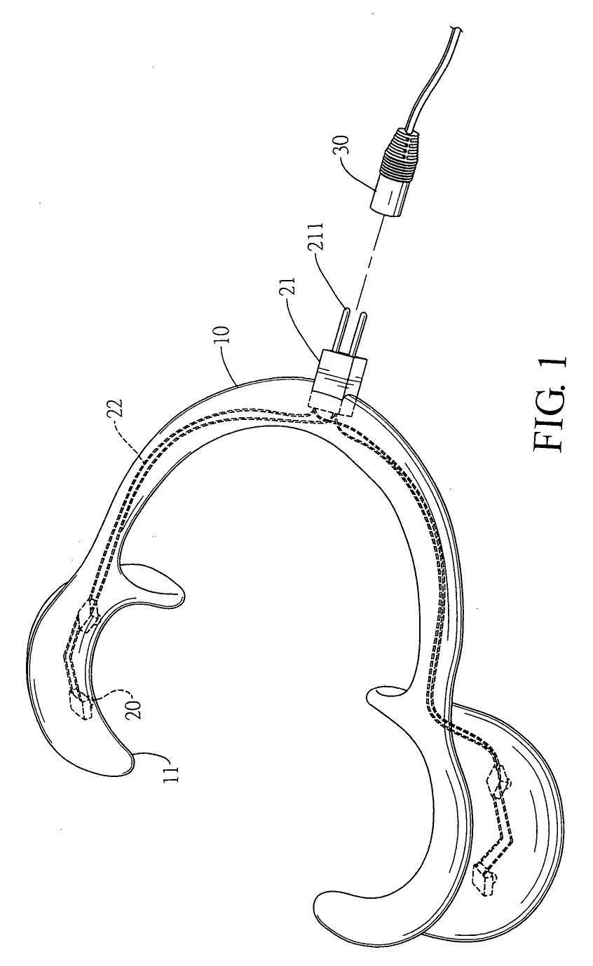

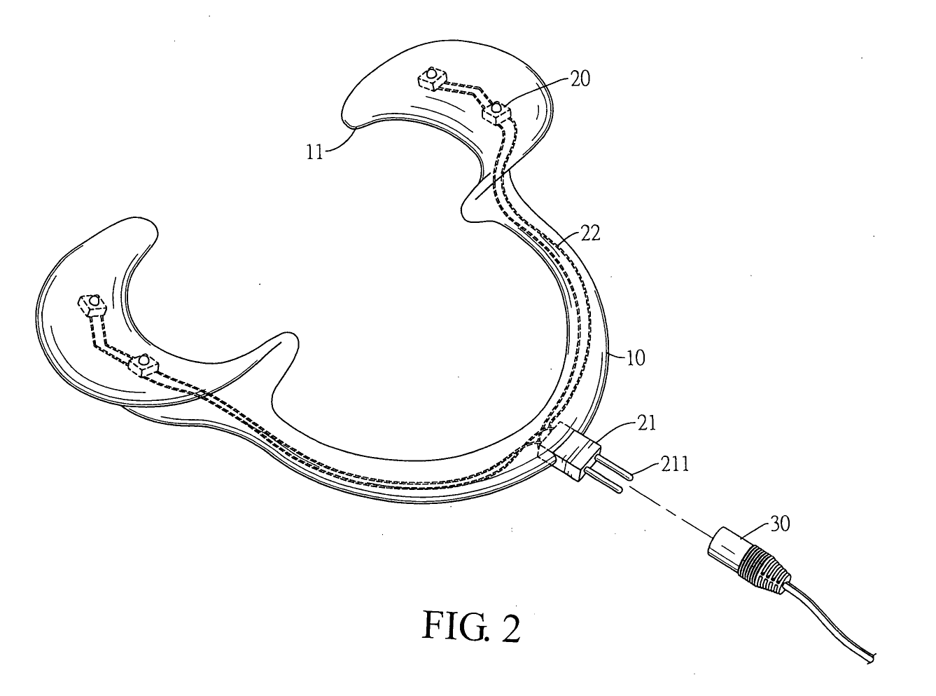

An illumination apparatus for illuminating a patient's oral cavity according to a first embodiment of the present invention is depicted in FIGS. 1 and 2. The illumination apparatus broadly includes a generally U-shaped sheet 10 having arcuate braces 11 defined at each of the two free ends thereof. Illumination devices 20 are operably coupled on one side of each arcuate brace 11. An electric connecter 21 is operably coupled with the illumination devices 20 via wires 22 at the closed end of the generally U-shaped sheet 10. In this embodiment, the illumination devices 20 comprise a plurality of light emitting diode (“LED”) light sources and the electric connecter 21 has outwardly protruding pins 211.

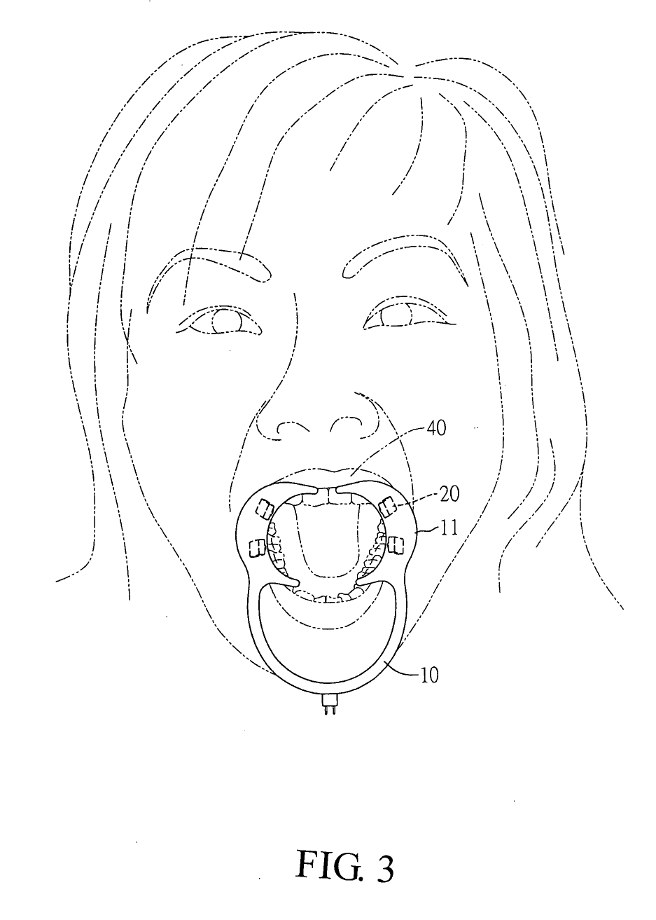

In use, as generally depicted in FIG. 3, an electric plug 30 can be coupled with the electric connecter 21 with the pins 211 inserted into electric plug 30. The other end of the electric plug 30 can be operably coupled with a battery set or a transformer, the type and usage of which are well-known to persons having ordinary knowledge in the related art.

The two arcuate braces 11 can be placed at the corners of the patient's mouth 40 to come into contact with the corners of the mouth 40. The generally U-shaped sheet 10, which expands outwardly, can make the mouth 40 expand toward both sides and further force the oral cavity to open. The illumination devices 20 emit lights to illuminate the patient's palatal and mandible at the same time. Thus the dentist can treat the teeth with still sufficient illumination in the oral cavity even if the external illumination is insufficient.

Alternatively, the present invention can be of disposable type. In other words, the illumination apparatus can be discarded after being used once or several times with sterilization so as to facilitate the dentist's use.

Further, the present invention can be also used in adverse circumstances of insufficient medical facilities such as field hospitals or temporary outdoor hospitals for serious natural disasters. The dentist can treat the patient with the present invention and simple medical appliances in the aforementioned circumstances.

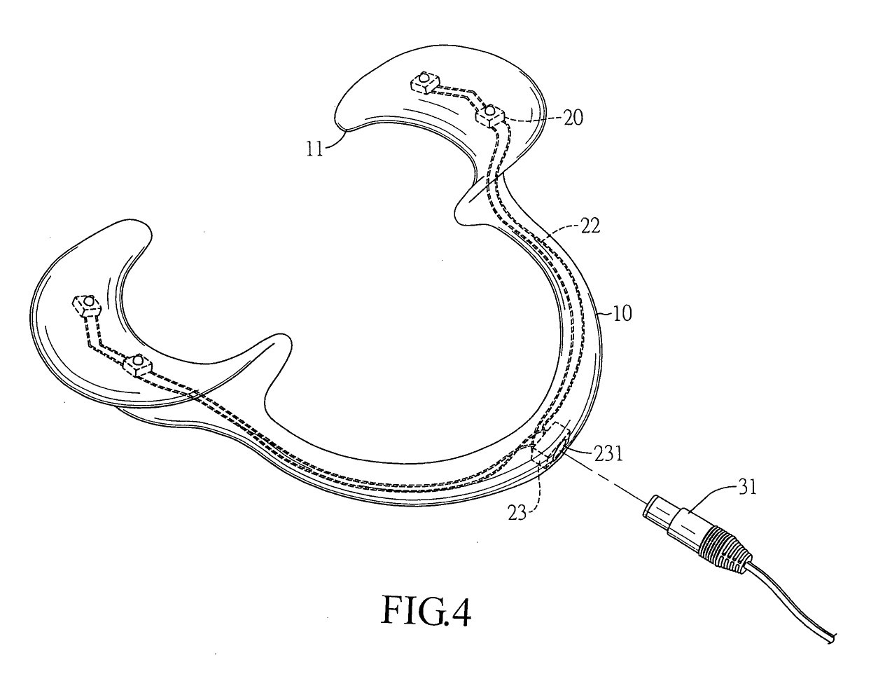

Referring to FIG. 4, a second embodiment of the present invention is depicted. In this embodiment, an electric connecter 23 has a conductive socket 231 for insertion of an electric plug 31.

In addition to the function of forcing the patient's oral cavity open, the illumination devices 20 can provide illumination in the oral cavity so that the patient can be well treated under the condition of sufficient illumination, and the types of the electric connecter 21, 23 and the electric plug 30, 31 can be modified according to various conditions.

Although the present invention has been described with reference to particular embodiments, one skilled in the art will recognize that changes may be made in form and detail without departing from the spirit and the scope of the invention. Therefore, the illustrated embodiments should be considered in all respects as illustrative and not restrictive.

What is claimed is:

1. An apparatus for illuminating an oral cavity, comprising:
a generally U-shaped member having two ends and an intermediate portion defined therebetween;
generally arcuate braces defined at each of the ends;
an illumination device operably coupled to at least one of the braces; and
an electric connecter operably coupled to the intermediate portion, the connecter being operably coupled to the illumination device with one or more wires.

2. The apparatus of claim 1, wherein the connecter has one or more pins protruding therefrom, the one or more pins insertable into an electric plug.

3. The apparatus of claim 1, wherein the connecter comprises a conductive socket for insertion of an electric plug.

4. The apparatus of claim 1, wherein the apparatus is disposable.

5. The apparatus of claim 1, wherein the illumination device comprises a plurality of LED light sources.

6. The apparatus of claim 5, wherein the connecter has one or more pins protruding therefrom, the one or more pins insertable into an electric plug.

7. The apparatus of claim 5, wherein the connecter comprises a conductive socket for insertion of an electric plug.

8. The apparatus of claim 5, wherein the connecter is operably coupled to a battery.

9. The apparatus of claim 5, wherein the connecter is operably coupled to a transformer.

10. The apparatus of claim 5, wherein the apparatus is disposable.

11. A method of illuminating a patient's oral cavity comprising:
providing a generally U-shaped member having two ends and an intermediate portion defined therebetween, generally arcuate braces being defined at each of the ends and an illumination device operably coupled to at least one of the braces, and an electric connecter operably coupled the generally U-shaped member, the connecter being operably coupled to the illumination devices with one or more wires;
operably coupling an electric plug to the connecter and positioning the braces proximate corners of the patient's mouth; and
illuminating the illumination device such that the device illuminates the patient's oral cavity.

12. The method of claim 11, wherein the illumination device generally simultaneously illuminates the patient's palate and mandible.

13. The method of claim 11, further comprising inserting pins included on the connector into an electric plug.

14. The method of claim 13, further comprising operably coupling the connector to a battery set.

15. The method of claim 13, further comprising operably coupling the connector to a transformer.

16. A method of making an illumination apparatus for an oral cavity, comprising:
providing a generally U-shaped member having two ends and an intermediate portion defined therebetween;
presenting generally arcuate braces at each of the ends;
operably coupling an illumination device to at least one of the arcuate braces; and
operably coupling an electric connecter to the intermediate portion, the connecter being operably coupled to the illumination devices with one or more wires.

17. The method of claim 16, wherein the electric connecter comprises outwardly protruding pins insertable into an electric plug.

18. The method of claim 16, further comprising presenting a conductive socket on the conductor for operable insertion of an electric plug.

19. The method of claim 16, further comprising operably coupling the connecter to a battery set.

20. The method of claim 16, further comprising operably coupling the connecter to a transformer.
